(19) United States
(12) Patent Application Publication
(10) Pub. No.: US 2007/0198017 A1
(43) Pub. Date: Aug. 23, 2007

(54) Soft tissue anchor

(75) Inventors: Alexander Tschakaloff, Pinneberg (DE); Randolf Von Oepen, Tuebingen (DE)

Correspondence Address: BILL R. NAIFEH, 12655 NORTH CENTRAL EXPRESSWAY, TENTH FLOOR, SUITE 1014, LB 55, DALLAS, TX, 75243, US

(73) Assignee: DEGIMA Medizinprodukte Gmbh, Pinneberg (DE)

(21) Appl. No.: 10/505,531

(22) PCT Filed: Feb. 21, 2003
(86) PCT No.: PCT/EP03/01796

§ 371 Date: Sep. 5, 2006

(30) Foreign Application Priority Data
Feb. 22, 2002 (EP) 02004017.6

(51) Int. Cl. A61B 17/04

(52) U.S. Cl. 606/73

(57) ABSTRACT

The present invention relates to an apparatus and method for connecting soft tissue or ligament to a bone using surgical suture. More specifically the present invention relates to an anchor for fixation into a bone wherein the amount of material for said anchor is minimised by leading the suture thread through a special passageway on the outside of said anchor.

### BACKGROUND OF THE INVENTION

In the field of surgery, there are occasions when a soft tissue or a ligament have to be reattached to a bone. In view of the high density of the bone compared to the relatively low density of soft tissue or ligament it is not feasible to use one kind of fixing element like a screw or a nail (which will work for the bone but not for the tissue) or suture (which will work preferably for the tissue and not the bone). Recently various types of threaded suture anchors have been developed for this purpose. Some suture anchors are designed for insertion into a pre-drilled hole. Other suture anchors are self-tapping. Examples for such anchor devices are disclosed in U.S. Pat. No. 5,156,616, U.S. Pat. No. 5,571,139 and U.S. Pat. No. 5,964,783.

These anchoring devices, which bare somehow a fixation means for a suture thread of desired length, are constructed of either a metal or any kind of plastics, including biodegradable materials. Depending upon the type of anchor the suture thread is either already attached to the anchor prior to implantation into the bone or will be connected to the anchor after implantation by using i.e. an eyelet. In either case, once the anchor and the suture thread are in place, the thread extending from the anchor is then used to fix the soft tissue or the ligament to the bone in a way enabling the tissue to reattach to the bone over the time.

In the utility model DE29717413 a thread anchor is described comprising a cylindrical threaded body with two or more holes passing through the body of the screw. These holes are used to hold a suture thread. For a doctor it is quite time consuming to use this device since he has to introduce the thread into one hole and then back again through another hole. Since these holes must comprise at least the diameter of a standard surgical thread, this device has a quite big outer diameter. In addition the doctor cannot use suture thread with prefixed needles with this device since it is not possible to pass the prefixed needles through the holes.

### DESCRIPTION OF THE INVENTION

For many applications, especially for cosmetic surgery in the face or for anchoring sutures in thin or weak bones, it is desirable to have an suture anchor with a very small diameter to reduce the diameter of the corresponding hole in the bone, in particular avoid cracks due to the drilling of the holes. In addition it is desirable to have a suture anchor on which the suture thread is easy and quick to mount. Finally it is desirable to have a suture anchor that could be used with suture thread comprising prefixed needles.

The suture anchor of the present invention overcomes disadvantages of the prior art as described above by providing a bone screw having a passageway for receiving the suture thread that does not extend inside the screw body, but extends around the body of the screw. Accordingly the bone screw comprises a smaller diameter and needs smaller holes for insertion into the bone. Finally the surgeon could use suture thread with prefixed needles, since he just has to place the suture thread around the screw into the passageway.

### BRIEF DESCRIPTIONS OF THE DRAWINGS

FIG. 1 is a plan view of a bone screw according to the present invention showing the thread portion and the non-threaded portion of the screw body and the passageway (1) for the suture thread extending around the screw body.

FIG. 2 is a cross-section of a bone screw according to the present invention showing the thread portion and the non-threaded portion of the screw body, the passageway (1) for the suture thread extending around the screw body and the proximal opening (6) for the use of a corresponding screw driver.

FIG. 3 is a view from the proximal end of a bone screw according to the present invention showing the proximal opening (6) for the use of a corresponding screwdriver.

FIG. 4 is a view from the distal end of a bone screw according to the present invention showing the central part of the passageway (4).

FIG. 5 (A to E) shows possible variations of the shape of the proximal opening of a bone screw according to the present invention.

### DETAILED DESCRIPTION OF THE DRAWINGS AND INVENTION

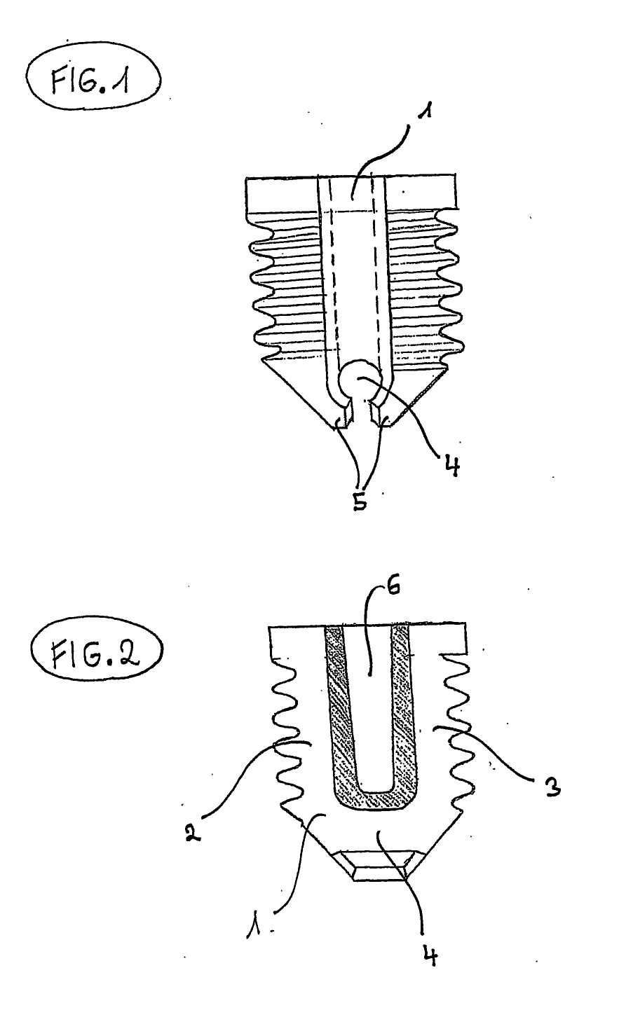

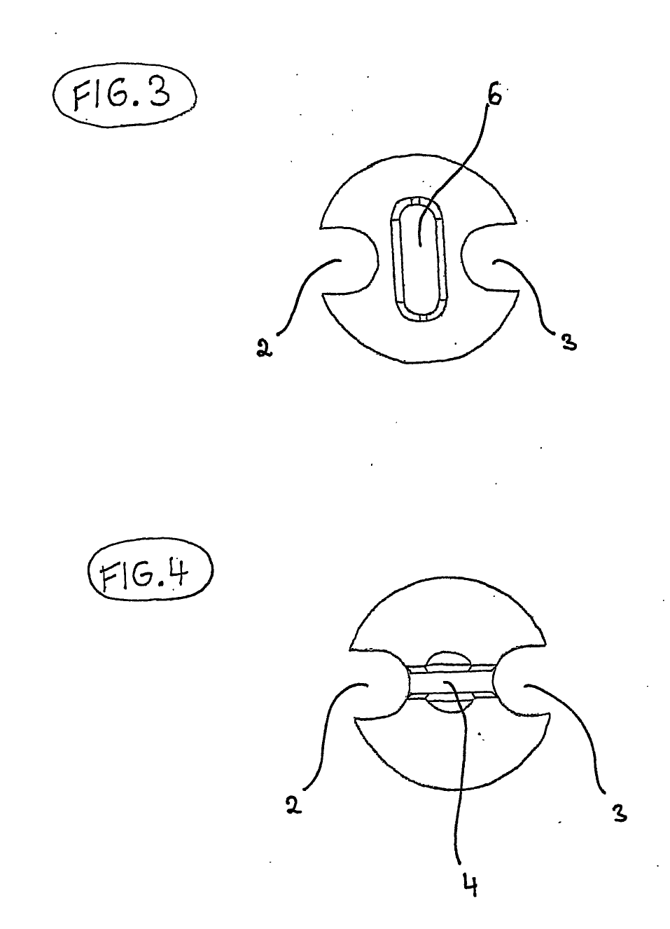

As shown in FIG. 1 the passageway (1) for receiving the suture thread leads along the screw body, going down on the front side, parallel to the longitudinal axis of the screw body, then around the distal tip and finally up on the other side. FIG. 2 shows a cross-section of a bone screw according to the present invention illustrating the passageway (1) for the suture thread extending around the screw body. The screw body could be cylindrical (as shown in FIG. 1 and FIG. 2) or it could be tapered, in particular conical, depending on the application of the suture anchor.

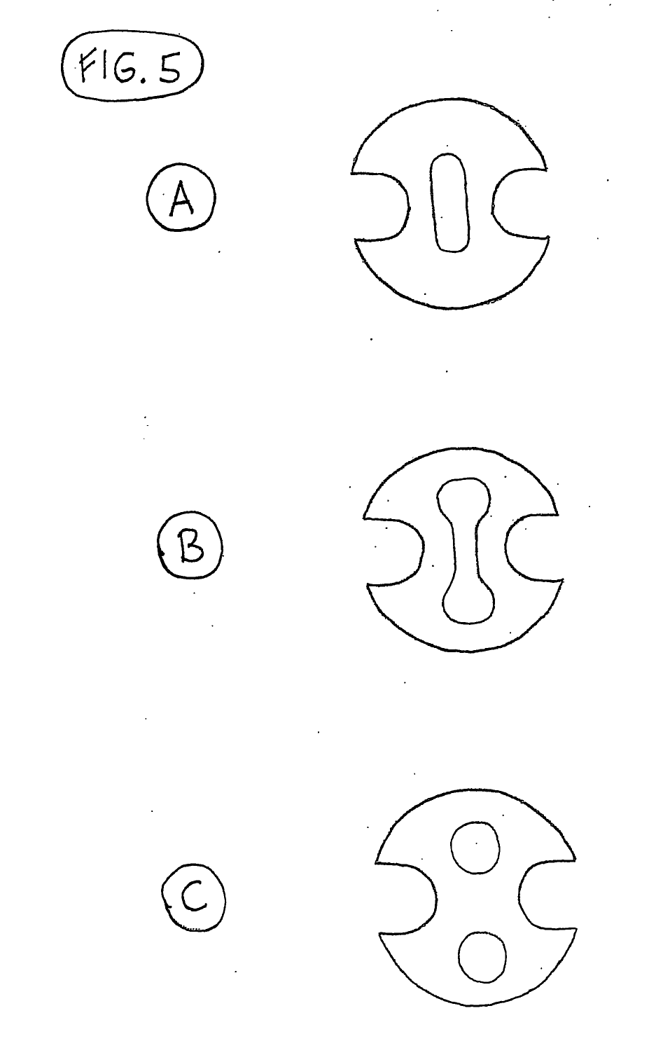

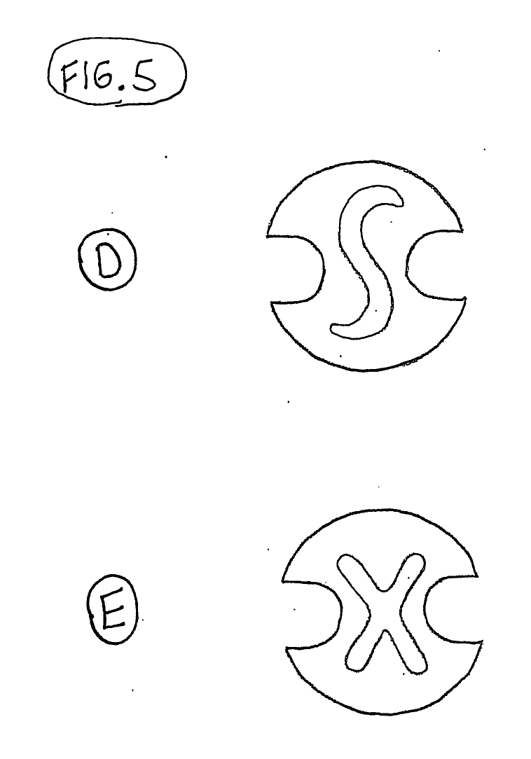

The side portions (2, 3) of the passageway, that are located parallel to the longitudinal axis of the screw, comprise a general circular cross-section as illustrated in FIG. 3. This cross-section is necessary for receiving the round suture thread properly and obtaining a movable thread even after insertion of the screw into the bone. The side portions (2, 3) are formed like tubes with longitudinal openings that are big enough to allow easy receive of the suture thread from the side prior to insertion of the screw anchor into the bone. FIG. 1, FIG. 2 and FIG. 4 show the distal end of the screw comprising the central portion (4) of the passageway. This part of the passageway has a circular cross-section as well to allow a movable thread. As illustrated in FIG. 1 the distal tip could comprise an additional element (5), so that the opening of the “tube” is narrowed. Therefore the thread could be placed into the central portion (4) of the passageway by applying some force to it. Advantageously the thread is subsequently fixed to the proximal end of the screw and could then easily be placed into the side portions of the passageway. At the same time the thread has still free movable ends so that the surgeon could adjust it according to his needs. The anchor including the attached suture thread could now easily be inserted into the bone. After insertion of the bone screw the proximal end flushes with the surrounding bone, which results in a smooth surface without influencing the surrounding soft tissue. The bone screw according to this invention could be either self-tapping or could be inserted in a pre-drilled hole dependent on the shape of its threaded body.

The bone screw illustrated in FIG. 1 comprises a threaded portion and a non-threaded portion of the screw body. The final drilling of the non-threaded proximal portion into the bone adds additional holding force to the one gained from tapping or inserting of a standard threaded screw, the screw gets “locked” into the bone.

FIG. 3 shows a view from the proximal end of the screw illustrating the passageway on two sides of the screw axis. In addition to that a proximal opening (6) is shown for receiving a screwdriver to insert the bone screw into the bone. The screw could comprise one opening as shown in FIG. 3 and in FIG. 2, it could also comprise two or more openings. These openings could have every suitable shape, in particular they could be shaped like a slit, a handle, a circle, a wave or a cross (see FIG. 5). It might be necessary to design a special screwdriver to fit exactly to the special proximal openings of the bone screws. On the other hand it could be possible to adapt the proximal openings to normal, commercial available screwdrivers. The material of the screw and/or the screwdriver could be chosen in a way the screw attaches very easy to the screwdriver via friction. This characteristic is preferred by the surgeon since it is very helpful if the comparable tiny screws are somehow attaching to the screwdriver. The screwdriver could also comprise an additional element to fix the free ends of the suture thread while the thread is attached to the bone screw and then inserted into the bone by the screwdriver.

The bone screw could be made from any suitable material, preferably any biocompatible material to minimise anti-inflammatory response. It could be manufactured from titanium, magnesium, thallium, stainless steel, Nitinol or any other suitable metals or any suitable polymer. Advantageous is the use of biodegradable polymers, since the bone screw is only needed for a limited time until the soft tissue is reconnected to the bone. Applicable are polysaccharides, polylactides, polyglycolids, polydiaxanoles or other suitable biodegradable polymers or according copolymers or blends. Degradation of implants made from biodegradable polymers often leads to irritation of the surrounding tissue due to the release of substances like lactic acids. Since the bone screw described in this invention needs less material than screws described in the prior art irritation of the surrounding tissue due to the degradation of the material is reduced. To limit the risk of irritation also special compounds could be incorporated into the screw material like antibiotics, immunosuppressiva, anti-inflammatory drugs or other suitable pharmaceuticals. If a biodegradable bone screw is used, incorporated bone growth factors could help to accelerate the ingrowth of the bone into the cavity left. A mixture of different compounds might be useful for special applications.

A coating to change the characteristics of the screw, e.g. the coefficient of friction, could cover the bone screw. If the screw has less friction, it is easier to insert the screw. Screws could also be covered by a biodegradable coating, eventually containing one or more compounds selected from the group of antibiotics, immunosuppressiva, anti-inflammatory drugs, bone growth factors or other suitable pharmaceutical to improve the acceptance of the implant.

For the manufacture of bone screws according to the present invention every suitable method could be applied. Bone screws made from polymers could be manufactured by injection moulding, by extrusion combined with mechanical finish, by producing a casted block, which is subsequently machined, or by using a block produced by polymerisation, which is subsequently machined. The manufacture process of injection moulding could be controlled in a way to result in an orientation of the polymer, so that the screw will relax after implantation and therefore will foreshorten and swell. This will then lead to additional anchoring of the screw into the bone. The density of the outer layer of the screw could be influenced by different injection parameters as well, which results in different degradation times. To manufacture a bone screw with incorporated compounds as discussed above bone screws could be incubated in a solution of the compound(s), so that there will be loaded a certain amount of compound into the screw. Dependent on its characteristics the compound could be added to the polymer solution or the polymer resin prior to manufacturing of the screw.

Another aspect of the invention is a method of attaching suture thread to bone that comprises the following steps. First, a bone screw is provided that comprises a screw body having a proximal and distal end, said body including exterior screw threads for inserting and retaining said body to the bone and a passageway extending around the body, said passageway including two side and one central portion, the portions being sized to receive a suture thread there through. Second, a suture thread is passed through the passageway of the body of the bone screw. A hole is eventually drilled into the bone and finally the bone screw is inserted into the bone.

It will be understood that the above-described device is only one example that illustrates the principle of the invention. Various modifications can be made by those skilled in the art without departing from the scope of this invention.

1. A bone screw for anchoring a suture thread to bone, comprising:
a screw body having a proximal and distal end, said body including exterior screw threads for inserting and retaining said body to the bone;
a passageway extending around the body, said passageway including two side and one central portion, the portions being sized to receive a suture thread there through.

2. The bone screw of claim 1, wherein the screw body is basically cylindrical.

3. The bone screw of claim 1, wherein the screw body is tapered.

4. The bone screw of claim 1, wherein the proximal part of the passageway comprises an element for attaching the suture thread in a way so that it is secured to the bone screw but could still move freely.

5. The bone screw of claim 1, wherein the screw body comprises a non-threaded portion at the proximal end.

6. The bone screw of claim 1, wherein the screw is constructed of a biocompatible material.

7. The bone screw of claims 1, wherein the screw is made from titanium, magnesium, thallium, stainless steel, nitinol or any other suitable metals.

8. The bone screw of claims 1, wherein the screw is made from a polymer.

9. The bone screw of claim 8, wherein the screw is made from a biodegradable polymer.

10. The bone screw of claim 9, wherein the screw is made from polysaccharide, polylactide, polyglycolid, polydiaxanole or other suitable biodegradable polymers or according co-polymers or blends.

11. The bone screw of claim 9, wherein the screw material has incorporated one or more compounds selected from the group of antibiotics, immunosuppressiva, anti-inflammatory drugs, bone growth factors or other suitable pharmaceuticals.

12. The bone screw of claims 1, wherein the screw is covered with a biodegradable coating.

13. The bone screw of claim 12, wherein said coating includes one or more compounds selected from the group of antibiotics, immunosuppressiva, anti-inflammatory drugs, bone growth factors or other suitablepharmaceuticals.

14. The bone screw of claims 1, wherein the screw is covered with a coating comprising a low coefficient of friction.

15. The bone screw of claims 1, wherein the screw is fixed to any kind of suitable suture thread before anchoring the screw into the bone.

16. The bone screw of claims 1, wherein said exterior screw threads are self-tapping.

17. The bone screw of claims 1, wherein the screw comprises at least one proximal opening for the use of a screwdriver to insert the bone screw into the bone.

18. The bone screw of claim 17, wherein the at least one proximal opening is shaped like a slit, a handle, a circle, a wave or a cross.

19. The bone screw of claim 17, wherein the screw is attaching to the screwdriver via friction.

20. A screwdriver for inserting a bone screw according to claim 17 into a bone comprising means for attaching to the at least one proximal opening of the bone screw.

21. The screwdriver according to claim 20 comprising an element to fix the suture threads attached to the bone screw prior to implantation during the process of inserting the bone screw into the bone.

22. A process to manufacture a bone screw according to claim 8 using injection molding.

23. A process to manufacture a bone screw according to claim 8 using extrusion combined with mechanical finish.

24. A process to manufacture a bone screw according to claim 8 using a casted block, which is subsequently machined.

25. A process to manufacture a bone screw according to claim 8 using a block produced by polymerisation, which is subsequently machined.

26. A process to manufacture a bone screw according to claim 11, wherein the compound is incorporated into the bone screw by incubating the bone screw with a solution of the compound.

27. A process to manufacture a bone screw according to claim 11, wherein the compound is added to the polymer prior to the manufacturing process of the bone screw.

28. The bone screw of claim 22, wherein the manufacturing process leads to an orientation of the polymer, so that the screw will relax after implantation and therefore will foreshorten and swell, which will lead to additional anchoring of the screw into the bone.

29. A method of attaching suture thread to bone, comprising the steps of:
providing a bone screw comprising a screw body having a proximal and distal end, said body including exterior screw threads for inserting and retaining said body to the bone and a passageway extending around the body, said passageway including two side and one central portion, the portions being sized to receive a suture thread there through,
passing a suture thread through the passageway of the body of the bone screw,
eventually drilling a hole into the bone,
inserting the bone screw into the bone.
